(19) United States
(12) Patent Application Publication
(10) Pub. No.: US 2009/0155842 A1
(43) Pub. Date: Jun. 18, 2009

(54) PROGNOSTIC ASSAY FOR METACHRONOUS COLORECTAL CANCER

(76) Inventors: Johannes Pieter Albert Baak, Stavanger (NO); Emilius Adrianus Maria Janssen, Stavanger (NO)

Correspondence Address: BROWDY AND NEIMARK, P.L.L.C.;624 NINTH STREET, NW, SUITE 300, WASHINGTON, DC, 20001-5303, US

(21) Appl. No.: 12/095,300

(22) PCT Filed: Nov. 22, 2006
(86) PCT No.: PCT/GB2006/004360

§ 371 Date: Nov. 17, 2008

(30) Foreign Application Priority Data
Nov. 28, 2005 (GB) 0524190.6

(51) Int. Cl. C12Q 1/08

(52) U.S. Cl. 435/40.52

(57) ABSTRACT

The invention provides a prognostic assay for metachronous colorectal carcinoma, which assay comprises obtaining a colorectal mucosa tissue biopsy sample taken from a human or non-human mammalian subject, and determining the average longest nuclear axis for a set of elongate nuclei in elongate cells in dysplastic tissue within said sample.

This invention relates to a prognostic assay for metachronous colorectal cancer, and a method of prognosis of metachronous colorectal cancer.

Colorectal cancer is responsible for a significant proportion number of all cancer related deaths. Early diagnosis is thus of great interest.

Colorectal cancer (carcinoma) is often preceded by a non-invasive neoplastic stage called colorectal adenoma (CRA). CRAs (polyps) can be discovered by colonoscopy; however the current evaluation criteria are not very accurate prognosticators of later-occurring (i.e. metachronous) colorectal carcinoma.

Colorectal carcinomas fall into two categories, synchronous and metachronous. With synchronous colorectal carcinoma, the time delay between adenoma and carcinoma is generally less than 24 months, the cancer develops generally in or close to the adenoma (typically from millimetres to less than 3 centimetres away) and the degree of dysplasia, i.e. abnormal cell morphology, correlates well with the risk of carcinoma. With metachronous colorectal carcinoma, the time delay between adenoma and carcinoma is generally greater than 24 months, the cancer develops at a distance of at least 3 centimetres away from the adenoma (often more than 5, particularly more than 8 centimetres), and the degree of dysplasia does not correlate well with the risk of carcinoma. While the incidence rate of metachronous cancer is relatively low, where CRAs are found and synchronous colorectal carcinoma does not develop or is ruled out, patient follow-up to check for metachronous colorectal cancer is time consuming, expensive and inconvenient and any test which allows a significant proportion of patients to be categorized as low or no risk can significantly reduce such burdens. There is thus a need for an assay with improved prognostic accuracy for metachronous colorectal cancer.

Meijer et al. in J. Pathology 184:63-70 (1998) reported on investigations into the prognostic value of certain features in rectosigmoid adenomas. In particular they investigated cell nucleus morphology (more particularly standard deviation of nuclear area (SDNA)) and the relative arrangement of cell nuclei in the epithelium in samples of such adenomas. While some correlation was found between development of metachronous colorectal cancer and the combination of low SDNA and cell crowding, the prognostic value was generally poor and the results have not been found to be repeatable.

We have now found a novel and highly accurate phenotypic prognosticator of metachronous colorectal carcinoma that can be detected in biopsy samples of colorectal mucosa at the CRA stage. This prognosticator is a cell type which occurs in the gland (the cylindrical indentations in the colorectal mucosal tissue responsible for releasing mucus into the gut).

Cells of this prognostic cell type are referred to herein as Monotonous Population of Elongated Cells (MPECs).

MPECS, as referred to herein, are elongate cells with elongate nuclei; their presence can be confirmed by measuring the median longest nuclear axis (LAM) of the elongate nuclei in elongate cells. Where the median LAM is above a specified threshold value, MPECs are considered to be present and the prognosis is that of risk of metachronous colorectal carcinoma. Below the threshold value the prognosis is that of no risk of metachronous colorectal carcinoma. As described in the Examples below, setting the threshold value at 14.9 μm resulted in all but one of the positives being correctly identified and about 50% of the negatives being correctly identified. Coupling the results with immunostaining increased the correctly identified negatives to about 80% and the correctly identified positives to 100%.

Thus, by assaying for MPECs in biopsy samples, it is possible to classify patients with a CRA as being at low-risk or high-risk of developing metachronous colorectal carcinoma. Low-risk patients can then be followed at elongated time intervals, perhaps at a frequency of 5 to 10 years, while high-risk patients may be followed more frequently, e.g. with colorectal endoscopy, and perhaps subjected to early systemic therapy, radiotherapy or surgery. Surgical removal of tissue identified at an early stage as being likely to develop into metachronous carcinomas gives an excellent prognosis for the patient. Currently CRA positive patients are followed at about 0.1 to 2 year intervals; with our new, more effective prognosticator of metachronous carcinoma, the burden on the physician is reduced, the suffering of the patient may be reduced, survival chances may be improved, and treatment costs may be reduced.

Thus viewed from one aspect the invention provides a prognostic assay for metachronous colorectal carcinoma, which assay comprises obtaining a colorectal mucosa tissue biopsy sample (typically from a CRA or polyp) taken from a human or non-human mammalian subject, determining the average longest nuclear axis for a set of elongate nuclei in elongate cells in dysplastic tissue within said sample, and optionally on the basis thereof assigning said subject as being at low risk or high risk of metachronous colorectal carcinoma, e.g. with patients having a median longest axis of at least 13.0 μm, more particularly at least 14.0 μm, especially at least 14.5 μm, more especially at least 14.9 μm being categorised as high risk.

The “average” longest nuclear axis may be determined using any desired statistical technique, e.g. it may be the mean, median or mode value. However the use of the median value is preferred.

MPECs are cells which are elongated and have elongated nuclei where the median of the longest nuclear axis (LAM) is at least 13 μm, particularly at least 14 μm, more particularly at least 14.5: m, especially at least 14.9 μm.

If desired, the variance of connectivity to 2 (C2V) of these cells, which is typically less than 80, particularly less than 50, may also be taken into account. C2V relates to the variation in the proximity of neighbouring nuclei, defined by minimum spanning tree analysis.

The median longest nuclear axis, and if desired C2V, should preferably be determined using the nuclei of a sample of at least 8 potential MPECs, more preferably at least 10, e.g. up to 50, to give the highest accuracy necessary in the biopsy.

The threshold value for the division between good and poor prognosis is dependent on the reliability of an assay; where, as here, the goal is primarily to divide patients into two groups, those requiring further attention or monitoring and those for whom monitoring may be made more infrequent, it is important to maximize the proportions of true negatives. Setting the LAM threshold too low does not achieve this; however setting it too high runs the risk of increasing the proportion of false negatives. By reducing operator bias by randomizing the selection of the nuclei contributing to the LAM value determined for a patient, we have found that it is possible to safely increase the threshold value up to 14 μm. Further improvements in nuclei selection, optionally combined with assessment of a secondary characteristic (e.g. immunostaining) may enable the threshold value to be further increased. The threshold value chosen will depend upon how it is computed, e.g. as the median, mode or mean. The values given herein are medians; however the equivalent values for means or modes may be computed in a straightforward fashion and the invention encompasses the use of alternatives to medians.

In the assay of the invention, the nuclei selected for longest axis measurement to determine the LAM are preferably selected randomly from the nuclei visible in the image. It is especially preferred that sampling is effected using systematic random sampling, e.g. as described by Gundersen et al. J. Microsc. 147:229-263 (1987), Cruz-Orive J. Microsc. 160:89-95 (1990), Roberts et al. J. Microsc. 171:239-253 (1993), and in particular Gundersen et al. J. Microsc. 193:199-211 (1989), the contents of which are incorporated by reference. Random selection, it has been found, increases the prognostic accuracy of the assay (and hence makes it possible to set as high as possible a threshold value for the divide between “prognosis is good” and “prognosis is bad”, i.e. between low or no risk and medium or high risk). In this regard it is important to note that the nuclei to be measured are not randomly selected from the whole mucosa of a CRA, but from a dysplastic region of the CRA's mucosa. The measurement procedure therefore generally starts with the delineation of the most dysplastic region from the CRA, in which the candidate MPECs are to be selected by random sampling of suitable nuclei within that region until a stable LAM value has been generated or until a preset number of sampling events or contributions to the LAM value have occurred. This delineated region is called the measurement area. The measurement area consists of very many fields of vision at the magnification that is suitable to properly visualize the nuclei. As the total number of fields of vision is too high for all to be inspected in an economically feasible assay, a random sample from these fields of vision has to be taken. The randomness of these sampled fields of vision is preferably achieved as follows. The first field of vision within the measurement area is selected at random. Then, all other fields of vision are distributed over the whole measurement area in such a manner that the whole measurement area is covered while at the same time the distances between the fields of vision is equal. As the first field of vision is selected randomly, all other fields are also spread randomly. The next step is to randomly select within each field of vision suitable nuclei (whether a nucleus is suitable or not is defined below). To further guarantee that these suitable nuclei are selected at random, a point grid is superimposed over each field of vision and only suitable nuclei hit by a point of that point grid are measured. Each sampling event thus typically involves seeking a nucleus in a randomly selected field, preferably using consistent seeking rules (e.g. first nucleus encountered while scanning a field in a particular fashion), followed by acceptance or rejection of the nucleus and LAM measurement (optionally omitted if the nucleus is rejected). Acceptance or rejection rules will generally be such as to include elongate nuclei cut longitudinally and to reject improperly cut nuclei and nuclei in deformed or otherwise uncharacteristic tissue. Sampling will generally proceed with sequential or batchwise selection of fields of vision. If desired, the procedure may be fully automated; however, in general, rejection may be operator effected during or after the sampling procedure. Typically the LAM value should be accepted only when the coefficient of error (CE, the ratio of the standard error of the mean of the determined LAM values to the mean of the determined LAM values) is below 8%, more preferably below or no greater than 5%, particularly below 3%.

While C2V could be determined manually, in practice it is preferred to use a highly automated determination procedure for syntactic structure analysis (SSA). SSA is a well established protocol which uses a video overlay system with an appropriate algorithm. Its use is described for example by Meijer et al in Anal. Quant. Cytol. Histol 14: 491-498 (1992), van Diest et al in Pathologica 87: 255-262 (1995) and Brinkhuis et al in Anal. Quant. Cytol. Histol. 19: 194-201 (1997). The variance of connectivity to 2 is a statistical measure of the variation in the tightness of neighbouring potential MPECs. Other equivalent statistical measures may be used and references herein to C2V being measured should be taken to encompass the measurement of such other statistical equivalents.

The investigation of the biopsy sample is preferably effected by microscopy using a section of the sample having on the inspected surface at least one gland, more preferably at least 10 glands, sliced transversely so as to expose potential MPECS sliced longitudinally in the gland walls. To facilitate identification of the MPECs, the section is preferably stained with a cell nucleus staining dye. Many such dyes are known in the art of histology. Typically however the nuclear sizes referred to herein may be determined using a standard haematoxylin-eosin stain of paraffin-embedded CRA material adequately fixed in buffered formaldehyde (typically 4% buffered formaldehyde), sectioned at 2-3 μm thickness, viewed with a 100× objective and with at least 15× after-magnification. Calculation of the lengths of the longest nuclear axes may be done by human measurement of the microscope images, or more preferably by computerized morphometric analysis (a well known procedure for which image analysis programs are commercially available).

Viewed from a further aspect the invention thus provides a biopsy tissue evaluation method comprising: obtaining a section of a colorectal mucosa tissue biopsy sample presenting a transverse section through at least one gland, preferably at least 10 glands, of CRA or polyp tissue; staining cell nuclei in said section; generating a microscope image of said section; selecting a set of longitudinally cut elongate cells (preferably randomly) with elongate nuclei in said image, preferably numbering at least 10, more preferably at least 15, particularly at least 20 such cells; and determining the average (e.g. median) longest nuclear axis (and optionally the variance of connectivity to 2) of said selected set of cells.

The glands are essentially cylindrical and when sliced transversely the resulting cross-sections are approximately oval with the potential MPECs appearing as longitudinally sliced elongate structures with their long axes perpendicular to the inner and outer surfaces of the gland. The potential MPECs selected are preferably selected from the flatter sides of the oval cross sections.

In a preferred embodiment of the invention, the selection of the candidate MPECs involves systematic random sampling in which a first field within the image, generally towards the centre of the section of the image containing the gland(s) (the “dysplastic area”), is selected at random whereafter a further plurality of fields, typically at least 100, more preferably at least 200, especially at least 250, typically 400 to 600, more particularly about 500, are selected by systematic random sampling. By using systematic random sampling, operator bias is avoided and the accuracy of prognosis is improved. Within each selected field a candidate MPEC is selected on the basis of inclusion and exclusion criteria. In many of the selected fields no suitable candidate MPEC may be found and the total number of selected fields is normally chosen to ensure that a total of at least 10, preferably at least 15, more preferably at least 20, e.g. at least 25, for example at least 50, candidate MPECs are selected. The number of candidate MPECs selected should desirably be sufficient that the coefficient of error in the determined median LAM is less than about 8%, more preferably less than about 5%, more particularly approximately equal to the optical resolution of the microscope which is generally about 0.25 μm.

The LAM for the selected candidate MPECs is measured and, preferably, the mean LAM is determined as a running mean, i.e. redetermined when each new candidate is measured. Once the running mean stabilizes, e.g. the coefficient of error is sufficiently low (e.g. <3%), and the total number of candidates measured is sufficiently high (e.g. >10), the selection and measurement may cease in order to save time. If measured in this way, a LAM of less than 14 μm correlates well with a good prognosis (i.e. no metachronous colorectal carcinoma) while a value above 14.9 μm correlates well with a poor prognosis (i.e. high risk of metachronous colorectal carcinoma). A value between 14 and 14.9 μm correlates well with a low risk of metachronous colorectal carcinoma.

The prognostic accuracy of the LAM value may be increased further by immunostaining a specimen of the tissue sample and counting immunostained (e.g. p16 stained) dysplastic epithelial nuclei. The staining agent used in this regard may be any of the agents known to bind to potentially cancerous cells, e.g. capable of binding to cell-surface epitopes more highly expressed on cancerous cells or cells in a growth or replication phase than to normal healthy somatic cells. Where such staining (e.g. with antibodies to p16) occurs, the positive predictive value of the LAM value, i.e. the correlation between high LAM and later cancer is increased significantly. The negative prediction value, i.e. the correlation between low LAM and no later cancer is already extremely high.

Biopsy tissue samples found to contain MPECS may also be investigated for chemical or biochemical markers for development of metachronous colorectal carcinoma and such biomarkers may be used as prognosticators for the development of metachronous colorectal carcinoma in patients, typically patients having colorectal polyps.

Such biomarkers may be explicitly identified and subsequently detected using specific binding partners (e.g. antibodies or antibody fragments, mRNA hybridizing probes, etc.). However it is possible to detect such biomarkers without knowing their chemical or biochemical identities using multivariate analysis of a data matrix obtained by hyphenated analysis of a tissue sample. In this regard by hyphenated is meant a technique in which a sample is separated chromatographically (e.g. using GC or LC) and the separated chromatographic sections are analysed using a spectrometric technique (e.g. MS or NMR). A prediction matrix may be generated using MPECs positive and MPECs negative samples and the prediction matrix may be applied to the data matrix for the unknown sample to provide an indication as to whether it is MPECs positive or MPECs negative. One version of this multivariate analysis technique is described in WO 02/03056 the contents of which are hereby incorporated by reference.

Thus viewed from a further aspect the invention provides an assay for MPECs cells in a colorectal mucosa tissue biopsy sample, said assay comprising analysing said sample for the presence or absence of biomarkers, particular proteinacrous biomarkers, for MPECs.

While multivariate analysis may be used in this assay method, the monoclonal antibodies to p16, p21, survivin, hTERT (telomerase) and beta-catenin, especially p16 and beta-catenin, more especially survivin and hTERT may be used to detect MPECS biomarkers. With p16, lack of expression is associated with no metachronous cancer if the LAM determined by microscopy is greater than 14.9 μm. In contrast, with beta-catenin, increased cellular expression of beta-catenin, especially in the cytoplasm, is a prognosticator for development of metachronous colorectal carcinoma. An increase of 40% or more relative to the expression levels in healthy cells can typically be taken as identifying a high risk patient.

More particularly, positive immunostaining of dysplastic epithelial cells in the sample with antibodies to p16 (5), p21 (10), nuclear beta-catenin (1), survivin (7.5) and hTERT (10) correlates with development of metachronous colorectal cancer, positive being staining of a certain minimum percentage of the visualized dysplastic epithelial cells in the sample, those minimum percentages typically being about the values in brackets in the previous sentence. Immunostaining antibodies usable in this regard are available commercially, e.g. from Novocastra Laboratories Ltd, UK and Santa Cruz Biotechnology Inc., California, US.

The antibodies mentioned above are generated by clones which are commercially available as follows:

| Antibody | Clone | Company | Dilution |
|---|---|---|---|
| Beta-catenin | clone 17C2 | Novocastra | 1:300 |
| hTERT | clone 44F12 | Novocastra | 1:100 |
| P16 | clone 6H12 | Novocastra | 1:25 |
| P21 | clone 4D10 | Novocastra | 1:25 |
| Survivin | clone D8 | Santa Cruz | 1:75 |

Multivariate analysis has shown that the combination of MPECs evaluation and immunostaining for survivin and/or hTERT represents the preferred basis for metachronous colorectal cancer prognosis.

While biomarkers for MPECs may particularly conveniently be assayed for in CRA or polyp tissue biopsy samples, other bodily tissues and fluids, including in particular blood and sputum, may likewise be assayed for such biomarkers, in particular protein biomarkers. Thus in effect the MPECs may serve as surrogate endpoint biomarkers (SEBs) for the early detection of adenomas which are high risk for development of metachronous colorectal carcinoma and the MPECs-SEBs may be used as the basis for proteomic analysis of the CRAS, or other body tissues or fluids. In particular, proteins found at unusually high or unusually low levels in MPECs as compared with equivalent gland cells in CRAs from patients not subsequently developing metachronous colorectal carcinoma may be assayed for in CRA tissue or other body tissues or fluids. As mentioned above, the chemical identities of such protein biomarkers do not need to be established and their existence may be determined by multivariate analysis and diagnostic reagents for them (e.g. antibodies) may be developed using chromatographically isolated but nonetheless nonidentified samples of them.

Thus viewed from a further aspect the invention provides a prognostic assay for metachronous colorectal carcinoma in a human or non-human mammalian subject, said assay comprising: contacting a tissue or fluid sample from said subject with a reagent capable of binding to or reacting with a protein biomarker (or set of biomarkers) the increased or decreased production whereof is characteristic of MPECs positive subjects; determining the level of binding or reaction by said reagent; and thereby assessing such subject as being of high risk or not high risk of development of metachronous colorectal carcinoma (e.g. by comparison with threshold values typical of such levels in such samples from subjects which have CRAs and which have or have not subsequently developed metachronous colorectal carcinomas).

Besides the assay of the invention, prognostic investigation for colorectal carcinoma should also desirably include monitoring of standard indicators, e.g. the number of detected colorectal adenomas (≧2 is an indicator of poor prognosis); and the location of the adenomas in the large intestine (if proximal, then the patient should be followed, if distal then the method of the invention may allow for patients to be identified as low risk).

It is thought that colorectal adenoma and as a result synchronous or metachronous, particularly metachronous, colorectal carcinoma, may result from bacterial infection, particularly from bacteria capable of expressing mammalian-cell-penetrating toxins, e.g. Shigella, E.coli, Actinobaccilus, Haemophilus, S. typhi, or H. pylori. Thus viewed from an alternative aspect the invention provides a method of treatment to combat colorectal dysplasia in a human or non-human mammalian subject in need thereof, said method comprising enterally administering to said subject an effective amount of an antibiotic.

In this method, the antibiotic will typically be administered orally or rectally. The determination of whether a subject is in need of such treatment will typically be by way of a positive result in an assay for the presence of MPEC, or for the presence of gut infection by bacteria capable of expressing cell-penetrating toxins, or as a result of a determination of the presence of dysplastic gut-wall tissue (e.g. by biopsy or endoscopy), or as a result of anal excretion of blood (e.g. as determined by assaying fecal matter). The antibiotic used may be any of the antibacterial agents commonly used to treat infection by such bacteria, in particular any of those used to combat H. pylori infection. The use of such antibiotics for the manufacture of a medicament for enteral administration to combat colorectal dysplasia, in particular colorectal carcinoma, also forms part of the present invention as do assays for the presence of gut infection by bacteria capable of expressing cell-penetrating toxins. In a further aspect the invention also provides the supply of an antibiotic for subsequent administration in such a method. In a yet still further aspect the invention provides a package comprising an antibiotic and instructions for its administration in such a method.

Examples of appropriate antibiotics for use in this regard include amoxicillin, clarithromycin, metronidazole, tetramycine, ampicillin, trimethoprim, sulphamethoxazole, nalidixic acid and ciprofloxin, optionally in combination with a cytoprotective agent, e.g. sucralfate or bismuth subsalicylate.

The invention will now be described further with reference to the following non-limiting Examples.

### EXAMPLE 1

### Biopsy Sample (Polyp) Preparation

The colorectal adenoma (CRA) sample should be representative for the whole polyp. If several polyps have been removed in one session, the one with the prognostically most unfavorable macroscopic and/or microscopic features should be selected for the analysis.

Cut the CRA longitudinally in slices of maximally 5 millimeter thickness.

Fix each of the CRA sample slices for 24 hours at 18° C. in 4% buffered formaldehyde.

Dehydrate routinely and embed in 56C paraffin.

Cut a 3 μm thick microscopic section from each sample slice.

Stain each sample section with haematoxylin and eosin (H&E). Select the subjectively most dysplastic slice, defined by architectural and cytonuclear abnormality.

For computerized morphometric analysis, use a representative Haematoxyllin stained (no eosin counterstaining) 3 μm microscopic section from this most dysplastic slice only.

In this microscopic section, an experienced pathologist should demarcate the most dysplastic area with a marker. This area (called the measurement area) should be minimally 1×1 millimeter and maximally 5×5 millimeter.

### EXAMPLE 2

### Assessment of Median Longest Nuclear Axis (LAM) And Mean Stratification Index (MSI)

The QPRODIT image analysis system (Leica, Cambridge, UK), or an equivalent system may be used for computerized morphometric analysis.

Open the QPRODIT program.

Select the “architecture” measurement module of the QPRODIT program.

Select the 100× objective. Take care that the projectives are such that they result in a screen magnification of 2000×.

For further analysis, select in the measurement area ten different fields of vision of longitudinally cut epithelium, if possible in adjacent crypts. Do not perform measurements in tangentially (obliquely, horizontally) cut epithelial sheets.

In these fields of vision, select only those nuclei that are longitudinally cut.

Select nuclei highest up in the crypt, but avoid surface epithelium. Select for measurement only those nuclei with a clearly defined nuclear membrane, preferably the most elongated nuclei.

Using the mouse, trace around the nuclear membrane of at least 10 adjacent nuclei in that field of vision.

Then, move to the next Field of Vision (FOV) and repeat the procedure, until 10 FOVs have been evaluated.

After ten fields of vision (in total at least 100 nuclei), assess the Median Longest Nuclear Axis (LAM) and the Mean Stratification Index (MSI).

Nuclei are selected according to a strict protocol:

1. Nuclei are focused at the largest diameter.

2. Only longitudinally cut nuclei of which the chromatin and the whole membrane (>90% around) can be discerned are measured. For LAM and MSI, tangentially or cross-cut nuclei are not selected.

3. Overlapping nuclei may be measured as long as the membrane of each nucleus can be unambiguously be discerned.

4. Mitotic figures, Paneth cells, lymphocytes and apoptotic bodies are not measured.

### EXAMPLE 3

### Assessment of Variance of Connectivity To 2 (C2V)

Open the QPRODIT program.

Select the “minimal spanning tree” measurement module of the QPRODIT program.

Select the 63' objective, with an appropriate projective, resulting in a screen magnification of 2000.

If in the measurement area, select ten different fields of vision, if possible in adjacent crypts. These crypts should be longitudinally cut, i.e. avoid tangentially/obliquely/horizontally cut crypts.

The video camera is rotated such that as many nuclei as possible are within the field of vision.

Using the mouse, one point is placed at the point of gravity of all nuclei (with a clearly defined nuclear membrane) within one epithelial sheet in the field of vision. Thus, both cross-cut and longitudinally-cut nuclei are measured, under the condition that the point can be placed without doubt in the gravity centre of the nucleus.

A minimum spanning tree, per field of vision, is created by the image analysis program when points have been placed in all relevant nuclei in that field of vision.

When measurements in all ten fields of vision have been performed, calculate the variance of the connectivity to 2 (C2V).

### EXAMPLE 4 (Best Mode)

### Measurement Protocol For LAM And P16

### The Quantitative Image Analysis System

Nuclear morphometric analysis of the representative H&E sections was performed with a motorized image analysis system (the “System”), see Rack et al. Int. J. Gynecol. Cancer 5:112-116 (1995) and Jannink et al. Breast Cancer. Res. Treatm. 36:55-60 (1995). The System consists of a personal computer with a mouse, a video overlay board, a colour camera mounted on a standard microscope equipped with an automated scanning stage, systematic random sampling software and measurement software. The live microscopic image is displayed in full colour on the monitor of the personal computer.

### Selection And Electronic Demarcation of the Measurement Area

The manually demarcated most dysplastic area was selected as follows:

1. Of all sections available, select the one with the most dysplastic area.

2. In that section, select the most dysplastic area.

3. In that most dysplastic area, avoid damaged, strongly inflamed or technically inadequate areas.

4. The thus selected area is called the Measurement Area.

5. Demarcate the Measurement Area with a black, water-resistant marker at the bottom of the microscopic glass slide of the microscopic specimen.

6. Put the thus demarcated slide on the scanning stage of the System. Using the System, at low magnification (objective 2.5×), electronically demarcate the measurement area.

### Systematic Random Sampling of Fields of Vision And Nuclei

The Fields of Vision and Nuclei were selected using rigid point-weighted systematic random sampling. This guarantees unbiased high reproducibility and stronger prognostic value (see Sorensen J. Microsc. 162:203-229 (1991) and Baak et al. Human Pathol. 25:80-85 (1994)).

### Selection of Fields OF Vision In the Measurement Area For LAM Measurement

The Fields of Vision (FOVs) for the measurements of the Median Longest Nuclear Axis (LAM) were selected in the measurement area as follows.

1. The number of FOVs in a measurement area was set at 250.

2. The System then selected a random point in the measurement area, which becomes the center of the first Field of Vision.

3. All other 249 fields of vision were systematically distributed by the System over the measurement area in such a manner that the distance between all FOVs was equal, and spread as much over the measurement area as possible.
This technique is called Systematic Random Sampling, as the first FOV is located at random and hence all other 249 FOVs are located at random in the Measurement Area.

### Definition of Nuclei To Be Selected For LAM Measurement

a. Inclusion Criteria of Nuclei For Measurement

Only select for measurement longitudinally cut nuclei in longitudinally cut glandular epithelial strips/crypts.

b. Exclusion Criteria For Nuclei
a. Overlapping or blurred nuclei where the individual nuclear membranes cannot be discerned separately
b. Small, round nuclei located towards crypt-bottom (which are tangentially cut)
c. Tangentially cut strips
d. Surface epithelium
e. Damaged strips, epithelium, or nuclei.
f. Strips <30 μm thickness (which means that the epithelium is artificially compressed!)

Longitudinally cut strips with a wavy luminal surface, or strips in sharp curves. However, the wavy strips are only excluded if the number of layers of nuclei, or the shape, changed. If they have the same form and size as nuclei in neighbouring normal longitudinal epithelium, the nuclei in wavy epithelium can be selected for measurement as well.

### Measurement of the Median Longest Nuclear Axis (LAM)

1. The measurement is started by giving the System a command to move to the first FOV.

2. In each FOV, the microscopic image is carefully focused.

3. Then, the image is “frozen”, and a point grid consisting of 20 white points appears overlaid over the microscopic image. The interpoint distance is 28 micrometer at specimen level.

4. To guarantee random unbiased selection of suitable nuclei, only nuclei hit by a point of the point grid are considered for measurement.

5. Only suitable nuclei are selected (see above for details).

6. The longest axis of the profile of a suitable nucleus is measured.

7. If other suitable nuclei in that FOV are hit by a point grid, that (these) nuclei are also measured.

8. Hereafter, the next FOV is selected by the system, until the last FOV. The system then moves the scanning stage to the first FOV.

9. The coefficient of error is continuously calculated and if it falls under 5%, the measurement is terminated but only if a minimum of 10 nuclei was measured. If it is above 5%, an additional 250 FOVS are selected at random by the system and more nuclei are selected and measured.

10. Intra- and inter-observer reproducibility of this method has proven to be very high (see Table 1 below).

TABLE 1
Inter operator variation

| | LAM (Micrometers) | | |
|---|---|---|---|
| Patient | Operator 1 | Operator 2 | Operator 3 |
| 1 | 14.3 | 13.7 | 13.8 |
| 2 | 14.8 | 14.7 | 14.2 |
| 3 | 11.6 | 10.5 | 11.6 |
| 4 | 14.5 | 14.7 | 14.4 |
| 5 | 16.8 | 16.5 | 16.3 |
| 6 | 18.2 | 18.3 | 18.9 |

### Quality Control of the LM Measurement

1. Store the measurement file and inspect the Running Mean graph for internal control.

2. Repeat another systematically randomly selected 250 FOVs (either by a new 250 FOVs selection, or by moving the scanning stage “3 clicks down and 2 clicks right”) for a total of 500 FOVs if:

a. Running mean is unstable (sharply descending/ascending)

b. The total number of nuclei selected for measurement are <10

c. The coefficient of error is >3%.

3. At the end of 500 FOVs, repeat at described under point 1 of this paragraph, if necessary select an additional 250 FOVs, and so on, until satisfactory results are obtained.

### Immunostaining For P16

Adjacent sections from the diagnostic biopsies were stained for p16 using immuno-histochemistry methods. Antigen retrieval and dilution of each of the antibodies were optimized before the study started. To guarantee uniform processing of the cases, all sections were made and immunostained at the same time. Paraffin sections of 4 micrometer thickness, adjacent to the H&E sections used for the CIN grade assessment, were mounted onto silanized slides (Dako, Glostrup, Denmark, S3002) and dried overnight at 37° C. followed by 1 hour at 60° C. The sections were deparaffinized in xylene and rehydrated in a graded series of alcohol solutions. Antigen retrieval was performed by pressure cooking in 10 mM TRIS/1 mM EDTA (pH 9.0) for two minutes at full pressure and cooled for 15 minutes at 18° C. Immunostaining was performed using an autostainer (DAKO, Glostrup, Denmark). TBS (Tris-buffered saline S1968 DAKO) was added at 0.05% and Tween 20 (pH 7.6) as the rinse buffer. Endogenous peroxidase activity was blocked by peroxidase blocking reagent S2001 (DAKO, Glostrup, Denmark) for 10 minutes and the sections were incubated with the monoclonal antibody p16 (clone 6H12), at dilution 1:25 (Novocastra, Newcastle upon Tyne, UK). DAKO antibody diluent S0809 was used and the immune complex was visualized by Peroxidase/DAB (ChemMate Envision Kit, DAKO, Glostrup, Denmark, K 5007) with incubation of Envision/HRP, Rabbit mouse (ENV) for 30 minutes and DAB+chromogen for 10 minutes. The sections were counterstained with Haematoxylin, dehydrated and mounted. Controls for the immuno-staining were performed using normal cervical tissue control sections and positive control sections on each slide stained, next to positive normal cell compartments (if available) within the test sections.

### Definition of Nuclei To Be Selected For P16 Measurement

Only dysplastic epithelial nuclei were counted, as either p16 positive or p16 negative. All other cells, tissue components or artifacts are ignored. Any dysplastic nucleus with some p16 positivity is regarded as positive. Using these criteria the sample point of the test grid (see below) could be p16 positive or p16 negative.

### Quantitation of the Percentage of P16 Immuno-Positive Cells

The p16 immunoquantitation was performed at a final microscope magnification of 200 (objective 240, numerical aperture 0.75, final screen monitor 831× magnification by means of the two-class immunoscoring module of the system with an electronic test grid graphically overlayed on the microscopic image and counters. In each field of vision one and the same of the four end-points of the smallest horizontal line grid available (point distance=at least 61.2 micrometer at specimen level), i.e. two horizontal lines is used for the point-counting. A sample of 350 points at the start of each measurement was used so that at least 200 nuclei were counted in each measurement area (average 220). At the magnification used, p16 positive dysplastic epithelial cells could accurately be distinguished from p16 negative dysplastic and other cells. The percentage was defined as [(p16 positive)/(p16 positive+p16 negative)]×100.

TABLE 2
P16 negative patients

| Number (n) | LAM range (μm) | Follow up status* |
|---|---|---|
| 1 | 10-10.9 | No cancer |
| 2 | 11-11.9 | No cancer |
| 3 | 12-12.9 | No cancer |
| 7 | 13-13.9 | No cancer |
| 20 | 14-14.8 | No cancer |
| 26 | 14.9-15.9 | No cancer |
| 9 | 16-16.9 | No cancer |
| 3 | 17-17.9 | No cancer |
| 1 | >18 | No cancer |

*at least 29 months, generally much longer.

Where the patients are broken down to p16 negative, p16 positive and p16 highly positive (Percentage 6 or greater), it can be seen how the proportion of true negatives and true positives may be enhanced using the assay method of the invention, especially if for highly positive cases the good/bad prognosis value for mean LAM is reduced from ≧14.9 μm to ≧14.0 μm.

TABLE 3
p16 Positive patients

| | | | Non Cancer |
|---|---|---|---|
| Number (n) | LAM range (μm) | Cancer Group | Group |
| 1 | 11-11.9 | 0 | 1 |
| 5 | 12-12.9 | 0 | 5 |
| 7 | 13-13.9 | 0 | 7 |
| 12 | 14-14.8 | 1* | 11 |
| 19 | 14.9-15.9 | 5 | 14 |
| 10 | 16-16.9 | 3 | 7 |
| 5 | 17-17.9 | 1 | 4 |
| 2 | >18 | 0 | 2 |

*LAM 14.5 μm, p16 highly positive

TABLE 4
p16 highly positive patients

| | | | Non Cancer |
|---|---|---|---|
| Number (n) | LAM range (μm) | Cancer Group | Group |
| 4 | 12-12.9 | 0 | 4 |
| 2 | 13-13.9 | 0 | 2 |
| 5 | 14-14.8 | 1 | 4 |
| 14 | 14.9-15.9 | 5 | 9 |
| 4 | 16-16.9 | 3 | 1 |
| 4 | 17-17.9 | 1 | 3 |

It should be noted that highly positive p16 staining, e.g. a prognostic divide line of 6% or greater, results in a false positive rate of 70%, in combination with a LAM threshold of 14.9: m this is reduced to 63%.

1. A prognostic assay for metachronous colorectal carcinoma, which assay comprises obtaining a colorectal mucosa tissue biopsy sample taken from a human or nonhuman mammalian subject, and determining the average longest nuclear axis for a set of elongate nuclei in elongate cells in dysplastic tissue within said sample.

2. An assay as claimed in claim 1 wherein said sample is from a colorectal adenoma or polyp.

3. An assay as claimed in claim 1 wherein said average is determined for nuclei in randomly selected areas of a section through said sample.

4. An assay as claimed in claim 1 wherein said average is determined with a coefficient of error below 8%.

5. An assay as claimed in claim 1 wherein said average is determined with a coefficient of error below 3%.

6. An assay as claimed in claim 1 wherein said average is the median.

7. An assay as claimed in claim 1 wherein a colorectal mucosa biopsy sample from said subject is subjected to immunostaining.

8. An assay as claimed in claim 7 wherein for a section through said sample the percentage of dysplastic cells that are immunostained is determined.

9. An assay as claimed in claim 7 wherein immunostaining is effected using an antibody to at least one of the group consisting of p 16, p21, beta-catenin, survivin and hTERT.

10. A biopsy tissue evaluation method comprising: obtaining a section of a colorectal mucosa tissue biopsy sample presenting a transverse section through at least one gland; staining cell nuclei in said section; generating a microscope image of said section; selecting a set of longitudinally cut elongate cells with elongate nuclei in said image; and determining the average longest nuclear axis of said selected set of cells.

11. A method as claimed in claim 10 wherein said set of cells is selected randomly.

12. A method as claimed in claim 10 wherein said average longest nuclear axis is determined on sufficient cells such that its coefficient of error is no greater than 5%.

13. A method as claimed in claim 10 wherein a colorectal mucosa biopsy sample from said subject is subjected to immunostaining.

14. An assay for MPECs cells in a colorectal mucosa tissue biopsy sample, said assay comprising analyzing said sample for the presence or absence of biomarkers for MPECs.

15. A prognostic assay for metachronous colorectal carcinoma in a human or non-human mammalian subject, said assay comprising: contacting a tissue or fluid sample from said subject with a reagent capable of binding to or reacting with a protein biomarker the increased or decreased production whereof is characteristic of MPECs positive subjects; determining the level of binding or reaction by said reagent and thereby assessing such subject as being of high risk or not high risk of development of metachronous colorectal carcinoma.
